(19) United States
(12) Patent Application Publication
(10) Pub. No.: US 2026/0053671 A1
(43) Pub. Date: Feb. 26, 2026

(54) OCULAR DEVICE FOR TREATING GLAUCOMA AND RELATED MINIMALLY INVASIVE GLAUCOMA SURGERY METHOD

(71) Applicant: UNIVERSITY HOSPITALS HEALTH SYSTEM, INC., Cleveland, OH (US)

(72) Inventor: Faruk ORGE, Cleveland, OH (US)

(73) Assignee: UNIVERSITY HOSPITALS HEALTH SYSTEM, INC., Cleveland, OH (US)

(21) Appl. No.: 19/104,733

(22) PCT Filed: Aug. 23, 2023
(86) PCT No.: PCT/US2023/072731

§ 371 Date: Feb. 19, 2025

Related U.S. Application Data
(60) Provisional application No. 63/400,780, filed on Aug. 25, 2022.

(51) Int. Cl. A61F 9/007

(52) U.S. Cl. CPC A61F 9/00781

(57) ABSTRACT

A bypass device is implanted into a body tissue via a minimally invasive surgery to provide fluid channels through the body tissue. The bypass device includes a base portion having opposite first and second ends, and flexed tines attached to the base portion configured in a flexed position relative to the base portion. The base portion includes flow spaces present from where the flexed tines are flexed relative to the base portion, the flow spaces permitting fluid flow through the bypass device. The bypass device further includes an anchor tine located adjacent to the second end of the base portion that aids in securing the bypass device in place after implantation. The bypass device may be implanted in the eye using a MIGS procedure for the treatment of glaucoma, whereby aqueous humor is permitted to flow through the flow spaces of the bypass device.

### TECHNICAL FIELD OF INVENTION

The technology of the present disclosure relates generally to an implantable ocular bypass device for treating glaucoma, and a related surgical procedure for implanting the ocular bypass device for use in the treatment of glaucoma.

### BACKGROUND

It is estimated that approximately three million people in the United States have glaucoma, and more than one hundred thousand people are blind from glaucoma. Glaucoma is the second leading cause of blindness in adult Americans age eighteen to sixty-five and the leading cause of blindness in African Americans. Glaucoma is an optic neuropathy, or a disorder of the optic nerve, that is characterized by an elevated intraocular pressure. An increase in intraocular pressure may result in changes in the appearance (“cupping”) and function (“blind spots”) in the visual field of the optic nerve. If the pressure remains high enough for a long enough period of time, total vision loss may occur.

The eye is a hollow structure that contains a clear fluid called aqueous humor. Aqueous humor is continuously produced in the posterior chamber of the eye by the ciliary body. The aqueous humor passes around the lens, through the pupillary opening in the iris and into the anterior chamber of the eye. Once in the anterior chamber, the aqueous humor drains out principally through a canalicular route that involves the trabecular meshwork and Schlemm's canal. The trabecular meshwork and Schlemm's canal are located at a junction between the iris and the cornea called the drainage angle. The trabecular meshwork is composed of collagen beams arranged in a three-dimensional sieve-like structure and lined with a monolayer of trabecular cells. The outer wall of the trabecular meshwork coincides with the inner wall of Schlemm's canal, which is a tube-like structure that runs around the circumference of the cornea.

The aqueous humor, while being filtered, travels through the trabecular meshwork into the Schlemm's canal, then from there through a series of collecting channels and reaches the episcleral venous system to be absorbed. In a healthy individual, aqueous humor production is approximately equal to aqueous humor outflow, and the intraocular pressure therefore remains fairly constant in the 10 to 21 mmHg range. High pressure develops in an eye because of an internal fluid imbalance. In glaucoma, the resistance through the canalicular outflow system is higher than normal causing reduced outflow, thereby causing an internal fluid imbalance and resulting in an increased pressure. In particular, the drainage angle formed by the cornea and the iris remains open, but the microscopic drainage channels in the trabecular meshwork are at least partially obstructed. Other forms of glaucoma may involve decreased outflow through the canalicular pathway due to mechanical blockage, inflammatory debris, or cellular blockage.

When the drainage system does not function properly, the aqueous humor cannot filter out of the eye at its normal rate. As the fluid builds up, the intraocular pressure within the eye increases. The increased intraocular pressure compresses the axons of the optic nerve, which carries vision signals from the eye to the brain, and also may compromise the vascular supply to the optic nerve. Damage to the optic nerve is painless and slow, and vision loss therefore can occur before a person is even aware of a problem.

There are various conventional ways of treating glaucoma. For example, eye and systemic medications are used to treat glaucoma by decreasing the production of aqueous humor or increasing the drainage from the eye.

Surgical treatment may be performed as a first line therapy or when medications fail to lower the intraocular pressure. For example, surgical procedures may be used to open up the anatomically closed drainage pathways of the aqueous humor to outside the eye. A trabeculectomy is a surgical procedure that creates a pathway for aqueous humor to escape to the surface of the eye. The anterior chamber is entered beneath the scleral flap and a section of deep sclera and trabecular meshwork is excised. Post-operatively, the aqueous humor passes through the resulting hole and collects in an elevated space (subconjunctival reservoir) beneath the conjunctiva. The fluid then is either absorbed through blood vessels in the conjunctiva or traverses across the conjunctiva into the tear film. A deficiency of such procedure is that as the formed bleb is extremely thin, many times the bleb can fail or erupt allowing a pathway for bacteria that normally live on the surface of the eye and eyelids to get into the eye.

Another surgical procedure involves the use of an aqueous tube shunt. A full thickness hole is made into the eye at the limbus, usually with a needle. The tube shunt is inserted into the eye through this hole and aqueous humor drains out to the surface of the eye. The tube is attached to a plate and this pate is placed underneath the extraocular muscles. The plate helps to create a reservoir again underneath the conjunctiva to where the aqueous humor drains. Many complications are associated with aqueous tube shunts. A thickened wall of scar tissue may resist outflow and limit the reduction in eye pressure. The bleb may not form quickly or not at all, resulting in an unrestricted flow through the shunt to the outer surface causing too low of an intraocular pressure that can damage the eye in different ways that could lead to loss of function and sight. As such shunts may erode through the overlying tissues creating an opening to the surface of the eye, a pathway is created for bacteria to get into the eye and endophthalmitis can occur.

Laser surgery is a surgical procedure to reduce the intraocular pressure and includes cyclophotocoagulation (reducing the production of aqueous humor by using a laser to burn the part of the eye that produces aqueous humor), iridotomy (use of a laser to make a hole in the iris to allow fluid to flow more freely in the eye), and trabeculoplasty (use of a laser to create holes in the drainage area of the eye to allow fluid to drain more freely). However, laser surgery is complex and suffers from a variety of deficiencies, including reduced effectiveness with time, inflammation, and related complications.

Accordingly, standard glaucoma surgeries are major surgeries that have significant deficiencies. While such surgeries are very often effective at lowering eye pressure and preventing progression of glaucoma, they have a long list of potential complications. To overcome such deficiencies, more advanced techniques have been developed which are commonly referred to as “minimally invasive glaucoma surgery” or MIGS. MIGS procedures work by using small-sized equipment and tiny incisions. While they reduce the incidence of complications, some degree of effectiveness is also traded for the increased safety.

The MIGS group of operations generally are divided into several categories: miniaturized versions of trabeculectomy; trabecular bypass operations; totally internal or supra-choroidal shunts; milder or gentler versions of laser photocoagulation; and ab-interno canaloplasty (ABiC). Generally, the MIGS procedures work by either bypassing the blocked trabecular meshwork (e.g., trabecular bypass operations and using supra-choroidal shunts), allowing the aqueous humor to drain to another potential space or by opening the Schlemm's canal and the collector channels (ABiC), or by decreasing the production of the aqueous humor (laser photocoagulation). Because of the advantage of MIGS procedures over more conventional treatments, efforts to improve MIGS procedures are on-going.

### SUMMARY OF INVENTION

The present application relates to an ocular implant bypass device and related minimally invasive glaucoma surgery (MIGS) for treating glaucoma with the ocular implant bypass device. The described MIGS technique includes piercing the trabecular meshwork to allow an opening to the trabecular meshwork and facilitate flow of aqueous humor to the Schlemm's canal.

The ocular implant bypass device includes a plurality of flexed tines. A leading flexed tine of the bypass device is first engaged to the trabecular meshwork, and then the ocular implant bypass device is pushed forward to create a trabecular meshwork opening or window, where the tip of the leading tine remains in the Schlemm's canal. The leading tine is pushed all the way to the back wall of the Schlemm's canal. At that point a second flexed tine of the bypass device is engaged by further pushing the proximal portion of the bypass device towards the trabecular meshwork using an introducer. Then both flexed tines are advanced via pushing the entire bypass device away from the introducer, and this pushing action also enlarges the opening in trabecular meshwork created by advancing the flexed tines. The created large opening in the trabecular meshwork now naturally corresponds to fluid spaces in a base portion of the ocular implant device, allowing aqueous humor to flow freely to the Schlemm's canal space to be drained through the collector channels. An anchor tine at the end of the bypass device is released from the introducer to act like a kickstand to sit over the trabecular meshwork. The pushing action stops when the kickstand anchor tine releases from the introducer tip, by which time an adequate amount of trabecular meshwork opening has been created, matched by the fluid spaces through the bypass device. In this manner, the fluid spaces of the bypass device naturally lie over the trabecular meshwork opening. The anchor tine prevents the bypass device from moving backwards naturally. The bypass device also can be easily removed by elevating the anchor tine from the trabecular meshwork to alleviate the kickstand operation, and sliding the flexed tines backwards towards the corresponding trabecular meshwork openings.

The ocular implant bypass device, therefore, may include one or more flexed tines extending from the base portion of the bypass device. Various parameters of the flexed tines may be varied and optimized for any particular use or body type, including length, shape, width, angle of the tines, size of the windows formed where the aqueous humor drains from the anterior chamber, and many other dimensions may vary depending on the intended use or patient circumstances. The curvature of the base portion of the bypass device is designed to fit the shape of normal curvature of the trabecular meshwork/Schlemm's canal complex. The curvature of the bypass device may be varied depending on the corneal diameter of a given patient. The backend, anchor tine of the ocular implant device has a bend to allow the back tip to be turned towards the trabecular meshwork, and the angle, length, width, and many other dimensions of the anchor tine again may be varied depending on the intended use or patient circumstances.

The ocular implant bypass device is a minimally invasive glaucoma surgery (MIGS) device as the bypass device minimally disrupts the tissues, being implanted through a very small incision, functions without disrupting surrounding tissues, and is easy to reverse. The implantation of the bypass device is performed using a specially designed introducer. The introducer includes a cannula at the tip where the bypass device is located and maintained at an initial retracted position. The introducer further includes an advancing plunger that also placed through the cannula and is positioned rearward relative to the bypass device. The advancing plunger is operated by pushing one or more levers of the introducer to advance the bypass device through the appropriate positions as described above.

An aspect of the invention, therefore, is an ocular implant bypass device that can be implanted into a body tissue to provide fluid channels through the body tissue, such as for example to provide fluid channels through the trabecular meshwork as part of a glaucoma treatment. In exemplary embodiments, the bypass device includes a base portion having a first end and a second end opposite from the first end; a first flexed tine and a second flexed tine attached to the base portion configured in a flexed position relative to the base portion, the first flexed tine being positioned closest to the first end of the base portion and the second flexed tine being positioned between the first flexed tine and the second end of the base portion; the base portion including a first flow space present from where the first flexed tine is flexed relative to the base portion and a second flow space present from where the second flexed tine is flexed relative to the base portion, the first and second flow spaces permitting fluid flow through the bypass device; and an anchor tine located adjacent to the second end of the base portion that aids in securing the bypass device in position during use.

Another aspect of the invention is a minimally invasive glaucoma surgery (MIGS) for treating glaucoma using a method of implanting a bypass device into the trabecular meshwork to define a fluid flow channel that permits aqueous humor to drain. In exemplary embodiments, the MIGS method includes the steps of: providing a bypass device according to any of the embodiments for bypassing a drainage system of an eye including the Schlemm's canal, trabecular meshwork, and collector channels; locating the bypass device within the cannula at an initial retracted position with an advancing plunger being positioned in the cannula rearward relative to the bypass device; forming a guide hole to access the Schlemm's canal at the trabecular meshwork of the eye; inserting the cannula with the bypass device and advancing plunger through the guide hole and positioning the cannula adjacent to the trabecular meshwork; operating the introducer operate the advancing plunger to advance the bypass device through the cannula from the initial retracted position to an intermediate position to expose the first and second flexed tines from the cannula; puncturing the trabecular meshwork with ends of the first and second flexed tines and forcing the flexed tines through the trabecular meshwork; operating the introducer to operate the advancing plunger to advance the bypass device through the cannula from the intermediate position to an extended position; wherein as the bypass device advances from the initial retracted position to the extended position, the flexed tines tear an elongated flow hole through the trabecular meshwork, such that when the bypass device reaches the extended position the elongated flow hole is aligned with the flow spaces of the base portion of the bypass device, and aqueous humor is permitted to flow through the flow spaces of the bypass device, and then through the aligned elongated flow hole through the trabecular meshwork to the Schlemm's canal so that the aqueous humor can drain; and retrieving the cannula with the wire while leaving the bypass device in place, wherein when the bypass device has reached the extended position and the cannula is removed, the anchor tine is positioned against an inner surface of the trabecular meshwork, and a curvature of the anchoring tine as positioned against the inner surface of the trabecular meshwork acts to generate a holding force to aid in maintaining the bypass device in place.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a drawing depicting a cross-sectional view of an eye.

FIG. 2 is a drawing depicting an enlarged cross-sectional view of an anterior chamber angle of the eye of FIG. 1.

FIGS. 3-5 are drawings depicting an exemplary bypass device from different viewpoints in accordance with embodiments of the present application.

FIG. 6 is a drawing depicting an exemplary introducer device that may be used for surgical implantation of the bypass device of FIGS. 3-5.

FIG. 7 is a drawing depicting a cross-sectional view of a portion of the exemplary introducer of FIG. 6.

FIGS. 8A-8D are drawings depicting different viewpoints of an exemplary first stage of a MIGS procedure in which the bypass device is in an initial retracted position.

FIGS. 9A-9D are drawings depicting different viewpoints of an exemplary second stage of a MIGS procedure in which the bypass device is in an intermediate position.

FIGS. 10A-10D are drawings depicting different viewpoints of an exemplary third stage of a MIGS procedure in which the bypass device is in a final extended position.

FIG. 11 is a drawing depicting an exemplary fourth stage of a MIGS procedure in which the bypass device is in the final extended position, with the cannula and stent being removed while the bypass device remains in position for use.

### DETAILED DESCRIPTION OF EMBODIMENTS

Embodiments will now be described with reference to the drawings, wherein like reference numerals are used to refer to like elements throughout. It will be understood that the figures are not necessarily to scale.

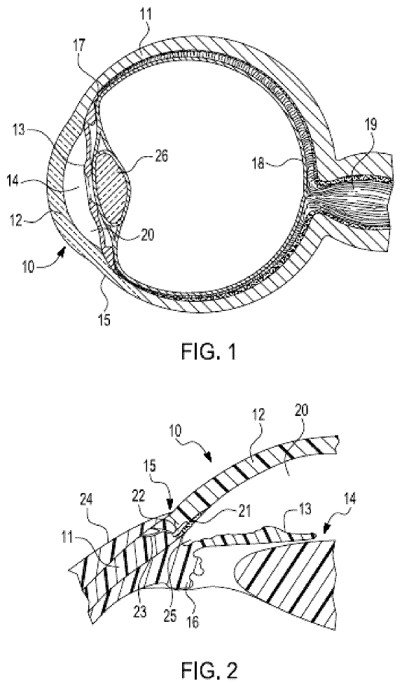

FIG. 1 is a drawing depicting a cross-sectional view of an eye 10, and FIG. 2 is a drawing depicting an enlarged cross-sectional view of an anterior chamber angle of the eye of FIG. 1, including the relative anatomical locations of the trabecular meshwork, the anterior chamber, and Schlemm's canal. Collagenous tissue known as sclera 11 covers the eye 10 except the portion covered by the cornea 12. The cornea 12 is a transparent tissue that focuses and transmits light into the eye, and the pupil 14 is the circular hole in the center of the iris 13 (colored portion of the eye). The cornea 12 merges into the sclera 11 at a juncture referred to as the limbus 15. The ciliary body 16 begins internally in the eye and extends along the interior of the sclera 11 and becomes the choroid 17. The choroid 17 is a vascular layer of the eye underlying the retina 18. The optic nerve 19 transmits visual information to the brain and is progressively destroyed by glaucoma as described above.

The anterior chamber 20 of the eye 10, which is bound anteriorly by the cornea 12 and posteriorly by the iris 13 and lens 26, is filled with the aqueous humor. As detailed above, aqueous humor is a fluid produced primarily by the ciliary body 16 and reaches the anterior chamber angle 25 formed between the iris 13 and the cornea 12 through the pupil 14. In a normal eye, the aqueous humor is removed through the trabecular meshwork 21. Aqueous humor passes through the trabecular meshwork 21 into Schlemm's canal 22 and through the aqueous veins 23 which merge with blood-carrying veins and into venous circulation. Intraocular pressure of the eye 10 is maintained by the intricate balance of secretion and outflow of the aqueous humor in the manner described above. Glaucoma is characterized by the excessive buildup of aqueous humor in the anterior chamber 20, which produces an increase in intraocular pressure that ultimately damages and then destroys the optic nerve.

The present application relates to an intraocular bypass device and related minimally invasive glaucoma surgery (MIGS) for treating glaucoma with the intraocular bypass device. In exemplary embodiments, the bypass device has a base portion and pronged features that extend from the base portion and are configured as flexed tines to create an open hole through the trabecular meshwork. The bypass device includes at least two such flexed tines. The bypass device further includes an anchor tine that extends from the base portion that aids in maintaining the bypass device in place after implantation. The base portion may have a curvature that approximates a curvature of the iridocorneal angle structures. The flexed tines are inserted through the trabecular meshwork at the desired locations. In particular, the flexed tines may be inserted at clogged locations of the trabecular meshwork as determined by a visualization technique. Once the bypass device is implanted, the anchor tine runs along an inner surface of the trabecular meshwork, which generates a holding force to aid in maintaining the bypass device in place. The base portion of the bypass device includes flow spaces present from where the flexed tines are flexed relative to the base portion, and the flow spaces permit fluid flow through the bypass device.

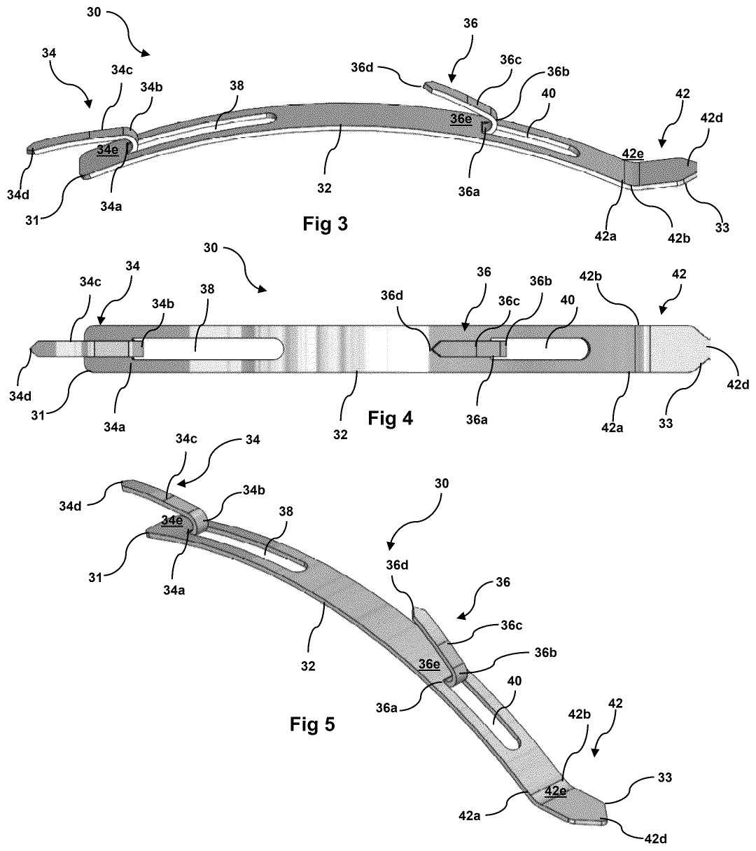

FIGS. 3-5 are drawings depicting an exemplary bypass device 30 from different viewpoints in accordance with embodiments of the present application. The bypass device 30 includes a base portion 32 that is formed with a curvature approximating the curvature of typical iridocorneal angle structures. The base portion defines flow spaces for the flow of fluid (e.g., aqueous humor) through the bypass device. As further detailed below, the bypass device is formed to include a plurality of pronged features configured as flexed tines that are attached to the base portion. The bypass device 30 also includes an additional anchoring tine that acts to generate a holding force to aid in maintaining the bypass device in place after implantation.

The bypass device may be formed using any suitable manufacturing process. Examples without limitation include laser cutting, photo-chemical etching, electrical discharge machining (EDM) or micro-machining, and micro-molding out of a plastic substrate. Materials that are used to form the bypass implant include, for example, nitinol, platinum, titanium, stainless steel, gold, silicon, PMMA, polyimide, or like materials. In an exemplary embodiment, the bypass implant material is nitinol and the shape is heat set in three stages to form the tines and curvature. A common base fixture may be used for all three heat setting stages, but in a different configuration or orientation, and the heat setting may be achieved at 525° C. for seven minutes per stage.

In the example of FIGS. 3-5, a plurality of flexed tines is configured with a flexed shape relative to the base portion 32. The base portion 32 has a first end 31 and a second end 33 opposite from the first end 31. In the depicted example of FIGS. 3-5, the bypass device 30 includes a first flexed tine 34 and a second flexed tine 36 that are spaced apart along the base portion 32. The first flexed tine 34 is positioned closest to the first end 31 of the base portion, and the second flexed tine 36 is positioned between the first flexed tine 34 and the second end 33 of base portion. Each of the flexed tines 34/36 includes an attachment portion 34a/36a where each of the flexed tines is attached the base portion, a curved portion 34b/36b that extends from and is curved relative to the attachment portion, a tine body 34c/36c that extends from the curved portion, and a pointed or arrow end 34d/36d that is used to puncture through the trabecular meshwork during implantation as further detailed below. With the curved portions 34b/36b being curved relative to the attachment portions, the flexed tines 34 and 36 extend from the base portion at respective acute angles 34e/36e relative to the base portion 32. The acute angles 34e and 36e may be the same or different. In addition, although the example of FIGS. 3-5 depicts two flexed tines 34 and 36 which may be suitable for typical circumstances, any suitable number of flexed tines may be employed as warranted for a particular circumstance based on the extent and location of blocked portions of the trabecular meshwork.

From where the flexed tines are flexed relative to the base portion 32, flow spaces are present in the base portion 32. In the example of FIG. 3 including the two flexed tines 34 and 36, a first flow space 38 is present from where the first flexed tine 34 is flexed relative to the base portion 32, and a second flow space 40 is present from where the second flexed tine 36 is flexed relative to the base portion 32. More generally, for whatever number of flexed tines are formed in the bypass device, a corresponding number of flow spaces is present from where the flexed tines are flexed relative to the base portion. As further detailed below, aqueous humor flows through the flow spaces of the bypass device to be drained through the trabecular meshwork.

The bypass device 30 also includes an anchor tine 42 formed at the second end 33 of the base portion 32. The anchor tine 42 includes an attachment portion 42a where the anchor tine is attached the base portion, a curved portion 42b that extends from and is curved relative to the attachment portion, and a pointed or arrow end 42d. In contrast to the flexed tines 34 and 36, an angle 42e of the of the curved portion 42b of the anchor tine 42 relative to the base portion 32 is an obtuse angle of less than 180°, and is preferably from 150° to 170°. As further detailed below, the obtuse angle of curvature of the anchoring tine acts to generate a holding force to aid in maintaining the bypass device in place after implantation.

A MIGS procedure to implant the bypass device generally is performed as follows. The leading flexed tine of the bypass device (i.e., flexed tine 34) is first engaged to the trabecular meshwork, and then the ocular implant device is pushed forward using an introducer to create a trabecular meshwork opening or window, where the tip of the leading flexed tine remains in the Schlemm's canal. The leading flexed tine is pushed all the way to the back wall of the Schlemm's canal. At that point the second flexed tine of the bypass device (i.e., flexed tine 36) is engaged by further pushing the proximal portion of the bypass device towards the trabecular meshwork using the introducer. Then both flexed tines are advanced via pushing the entire bypass device away from the introducer, and this pushing action also enlarges the opening in trabecular meshwork. The created large opening of the trabecular meshwork now naturally corresponds to the fluid spaces 38 and 40 in the base portion 32 of the bypass device, allowing aqueous humor to flow freely to the Schlemm's canal space to be drained through the collector channels. The anchor tine 42 at the second end of the bypass device is released from the introducer to act like a kickstand to sit over the trabecular meshwork. The pushing action stops when the kickstand anchor tine releases from the introducer tip, by which time an adequate amount of trabecular meshwork opening has been created, matched by the fluid spaces through the bypass device. The anchor tine prevents the ocular implant device from moving backwards naturally. The bypass device also can be easily removed by elevating the anchor tine from the trabecular meshwork to alleviate the kickstand operation, and sliding the flexed tines backwards towards the corresponding trabecular meshwork openings.

The ocular implant bypass device may be implanted using a minimally invasive glaucoma surgery (MIGS) for treating glaucoma with the bypass device. A MIGS procedure to implant the bypass device may include locating the bypass device within a specially designed introducer, and in particular within a cannula of the introducer, at an initial retracted position. An advancing plunger of the introducer is also placed through the cannula and is positioned rearward relative to the bypass device. The advancing plunger is operated by pushing one or more levers of the introducer to advance the advancing plunger through the cannula, thereby in turn advancing the bypass device through the appropriate positions as described above.

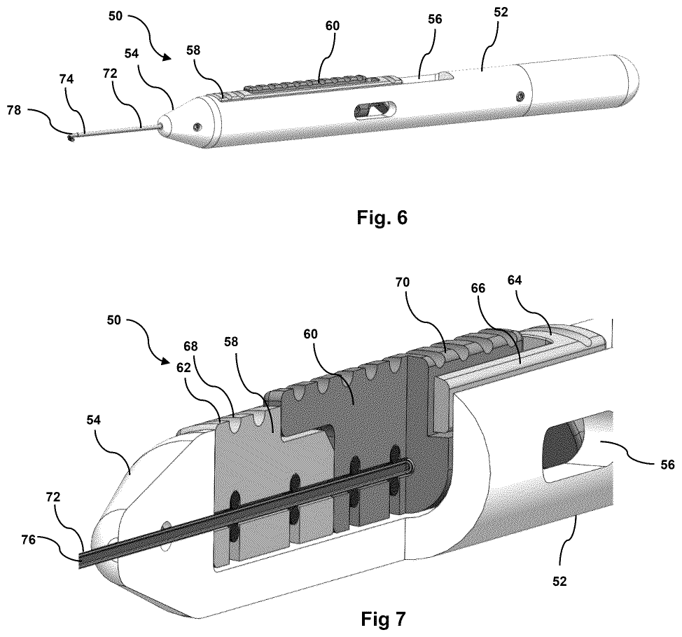

FIG. 6 is a drawing depicting a perspective view of an exemplary introducer device 50 that may be used for surgical implantation of the bypass device 30, and FIG. 7 is a drawing depicting a cross-sectional view of a portion of the introducer device 50 of FIG. 6. The introducer device 50 includes a body 52 that ends in a forward nose 54. The body 52 defines a recess 56 into which there are positioned a first slider 58 and a second slider 60. As seen particularly in the closeup viewpoint of FIG. 7, the first slider 58 has a front portion 62, a rear portion 64, and a bridge portion 66 that spans across the second slider 60 to connect the front and rear portions (the entire bridge is shown in FIG. 6). The first and second sliders each respectively may be configured with a ribbed upper edge 68 and 70. The ridged upper edges provide an enhanced surface for interaction with the hand of the surgeon, such as permitting easy movement of the sliders by the surgeon with a thumb forcing against the sliders along the ridged upper edges. The introducer 50 further includes a cannula 72 that extends through at least a portion of the first and second sliders 58 and 60, and a forward end 74 of the cannula 72 further extends through the forward nose 54 of the introducer device 50. The introducer further includes a guide plunger 76 that is threaded through the cannula 72 and anchored within the first and second sliders.

The forward end 74 of the cannula 72 may be fitted with a stent 78. The advancing plunger 76 is threaded through the cannula 72 and into the stent 78. The bypass device 30 is placed within the stent 78 with the advancing plunger 76 being positioned rearward relative to the bypass device 30. As referenced above, the base portion 32 of the bypass device 30 has a curvature approximating the curvature of typical iridocorneal angle structures, and therefore for use in implanting the bypass device 30, the stent 78 may have a commensurate curvature that also approximates such curvature of typical iridocorneal angle structures.

The MIGS procedure includes initially making an incision in the side of the eye cornea, and accessing the Schlemm's canal through a small guide hole made at the trabecular meshwork. A viscoelastic (gel-like) material may be introduced into the anterior chamber of the eye to maintain spacing within the anterior chamber suitable for the implantation. This allows the Schlemm's canal and the collector channels to be reopened, and also lubricates and expands the Schlemm's canal. The viscoelastic material may include a visualization agent which may be a dye, such as for example fluoresceine, tripan blue, or other suitable dye, or a physical visible agent such as micro-bubbles. The visualization agent may be visualized using any suitable imaging technique, such as for example optical coherence tomography or ultrasound bio-microscopy. Imaging of the visualization agent flowing through the aqueous humor removal system allows the Schlemm's canal, trabecular meshwork, and the collector channels to be viewed in great detail, which allows the surgeon to further identify the clogged areas of the trabecular meshwork/Schlemm's canal/collector channels versus open channels to ascertain an optimal location for insertion of the bypass device to bypass the blocked area.

The introducer device 50 is manipulated by the surgeon to move the cannula 72 though the guide hole to the location where the bypass device 30 is to be implanted at the trabecular meshwork. The cannula 72 including the stent 78 and bypass device 30 is guided along the anterior chamber curvature of the eye for proper placement for bypassing a blocked portion or portions of the trabecular meshwork. The cannula further may be used to introduce substances into the trabecular meshwork and/or Schlemm's canal such as glaucoma medications, anti-inflammatory agents, antibiotic releasing pellets, and the like for further success in intra-ocular pressure reduction, and for prevention of inflammation and related complications and infections. For example, a separate guide wire with a syringe-type tip may be threaded through the cannula beneath or otherwise adjacent to the bypass device. The guide wire with the syringe-type tip may be used for introducing fluids and other substances referenced above into the trabecular meshwork and/or Schlemm's canal.

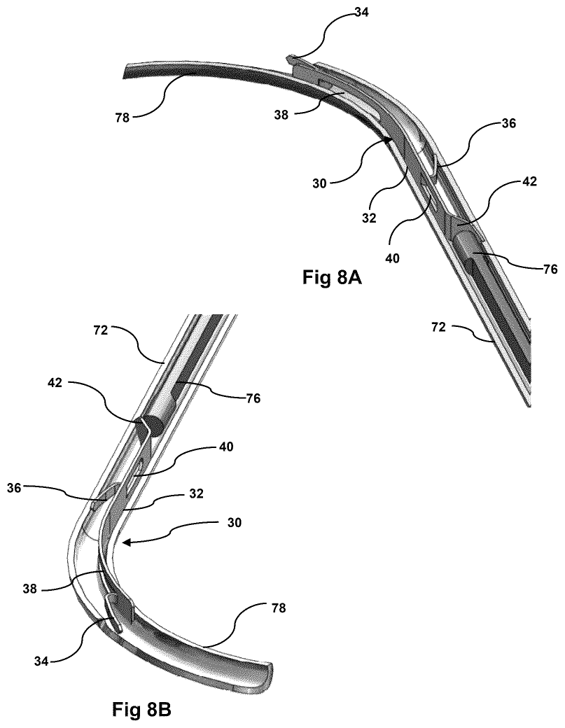

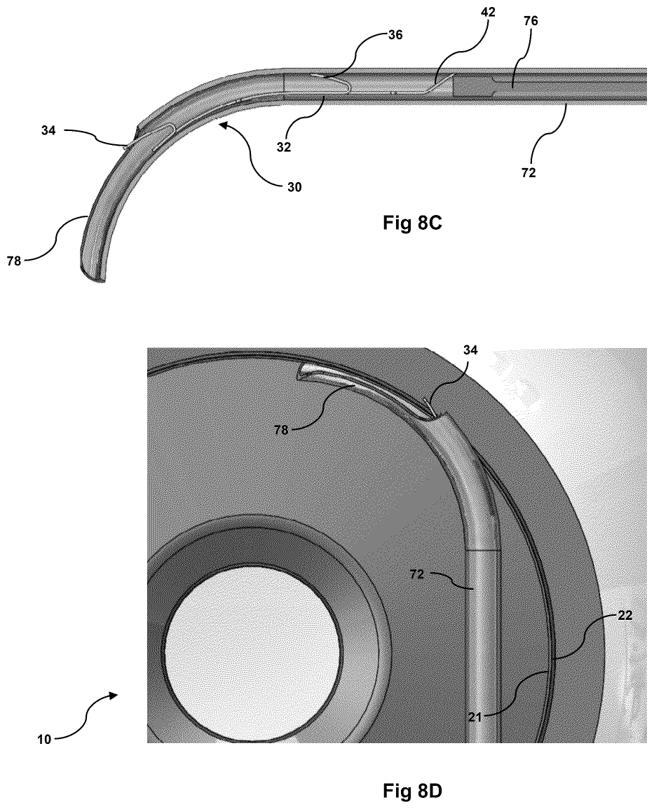

FIGS. 8-11 illustrate an exemplary MIGS procedure that employs the introducer device 50 for surgical implantation of the bypass device 30 into an eye. In particular, FIGS. 8A-8D are drawings depicting different viewpoints of an exemplary first stage of a MIGS procedure in which the bypass device 30 is in an initial retracted position. During this first stage in which the bypass device is in the initial retracted position, the bypass device 30 is encompassed fully or near fully within the stent 78. The advancing plunger 76 of the introducer has been threaded through the cannula 72 toward the stent 78, whereby the advancing plunger 76 is positioned rearward relative to the bypass device 30. The leading flexed tine 34 of the bypass device is first engaged to the trabecular meshwork 21 of the eye 10 oppositely from the Schlemm's canal (see particularly FIG. 8D).

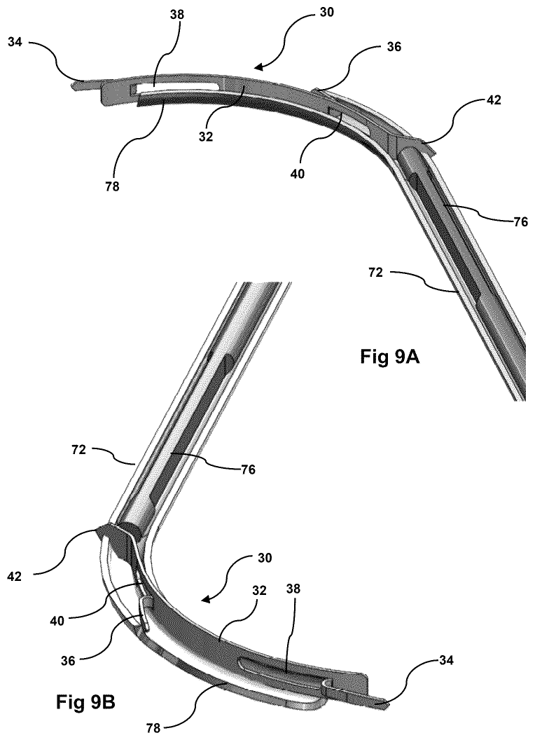

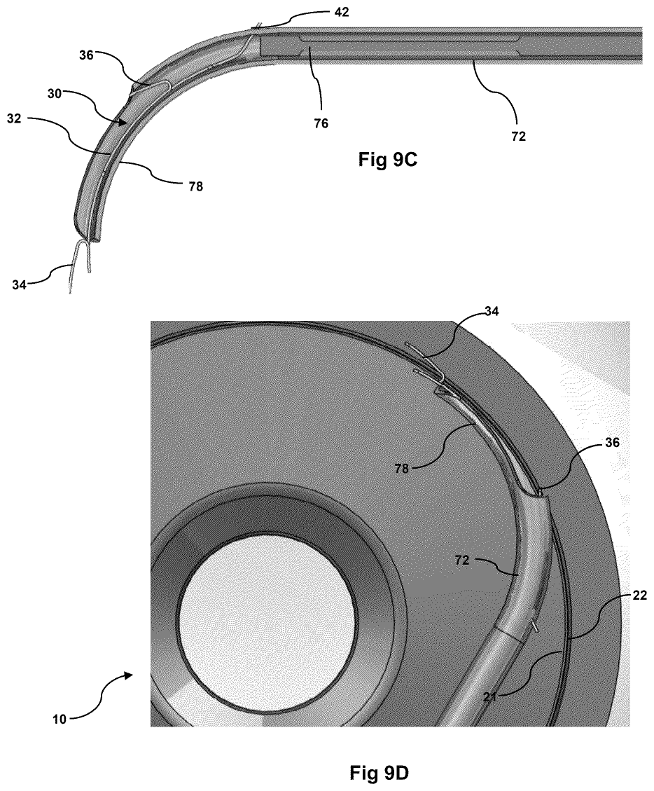

FIGS. 9A-9D are drawings depicting different viewpoints of an exemplary second stage of a MIGS procedure in which the bypass device is in an intermediate position. With further reference to FIGS. 6 and 7, during such second stage the surgeon operates the introducer device 50 by advancing the first slider 58 from a first rearward position to a first forward position. By advancing the first slider 58, the first slider advances the advancing plunger 76 forward which, in turn, advances the bypass device 30 further through the stent 78 from the initial retracted position to an intermediate position. In this manner, the second tine engages with the trabecular meshwork 21. The surgeon then punctures the trabecular meshwork 21 with the arrow ends 34d/36d of the flexed tines 34 and 36 and forces the flexed tines through the trabecular meshwork. More specifically, as referenced above in connection with FIG. 3, the flexed configurations of the tines 34 and 36 have the curved portions 34b/36b at acute angles relative to the base portion 32. Such curvature essentially forms flat springs that each has an associated spring force. The surgeon applying pressure of the flexed tines 34 and 36 against the trabecular meshwork tissue tends to a compress the flexed tines 34 and 36 toward the base portion 32 and against the spring force created by the curved portions 34b/36b of the flexed tines. The opposing spring force thereby aids the surgeon to push the arrow ends 34d/36d of the flexed tines 34 and 36 through the trabecular meshwork. During this second stage, therefore, the second flexed tine 36 of the bypass device is engaged by further pushing the proximal portion of the bypass device towards the trabecular meshwork using the introducer. Then both flexed tines are advanced via pushing the entire bypass device away from the introducer, and this pushing action also enlarges the opening in trabecular meshwork.

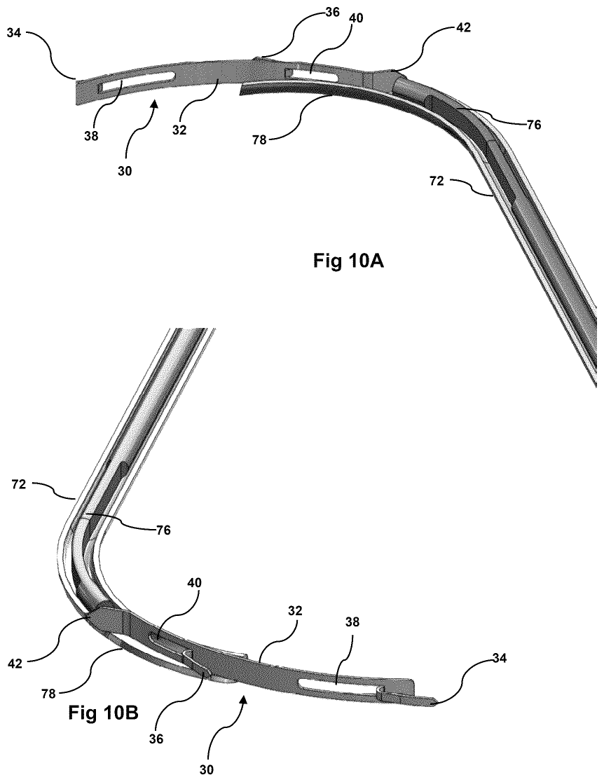

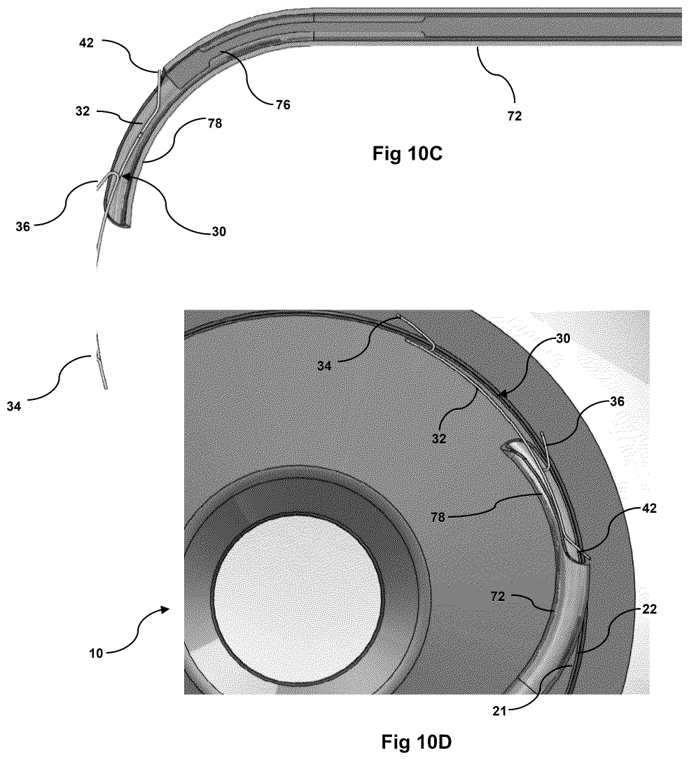

FIGS. 10A-10D are drawings depicting different viewpoints of an exemplary third stage of a MIGS procedure in which the bypass device is in a final extended position. During the third stage, the surgeon further operates the introducer device 50 by advancing the second slider 60 from a second rearward position to a second forward position. By advancing the second slider 60, the second slider further advances the advancing plunger 76 which, in turn, further advances the bypass device 30 through the stent 78 from the intermediate position the extended position. As referenced above, as the bypass device advances from the intermediate position to the extended position, the flexed tines tear an elongated flow hole through the trabecular meshwork 21. The result is that when the bypass device 30 reaches the final extended position, an elongated flow hole of the trabecular meshwork is aligned with corresponding flow spaces 38 and 40 through the base portion 32 of the bypass device 30. With this configuration, aqueous humor is permitted to flow through the through the base portion 32 via the flow spaces 38 and 40 of the bypass device 30, and then through the aligned elongated flow hole through the trabecular meshwork and into the Schlemm's canal 22 so that the aqueous humor can drain. During this third stage, the continued pushing action of the introducer further enlarges the opening in trabecular meshwork. The pushing action stops when the anchor tine 42 releases from the introducer. The created large opening of the trabecular meshwork now naturally corresponds to the flow spaces 38 and 40 in the base portion 32 of the bypass device, allowing aqueous humor to flow freely to the Schlemm's canal space to be drained through the collector channels. When the anchor tine 42 at the second end of the bypass device is released from the introducer, the anchor tine acts like a kickstand to sit over the trabecular meshwork. The anchor tine thereby prevents the ocular implant device from moving backwards naturally.

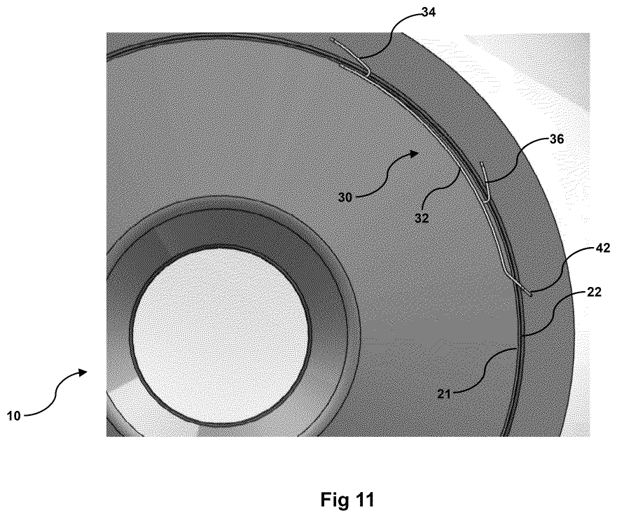

FIG. 11 is a drawing depicting an exemplary fourth stage of a MIGS procedure in which the bypass device is in the final extended position, with the cannula and stent being removed while the bypass device remains in position for use. Once the bypass device 30 is properly positioned, the introducer 50 is pulled away from the eye, thereby retrieving the advancing plunger 76 and cannula 72 while leaving the bypass device 30 in place. When the bypass device 30 has reached the final extended position and the cannula with the advancing plunger is removed, the anchor tine 42 is positioned against an inner surface of the trabecular meshwork 21. In particular, due to the curvature of the curved portion 42b of the anchor 42, the anchor tine extends along the inner surface of the trabecular meshwork 21 against and in opposition to the curvature of such inner surface. As a result, a force interaction between the anchor tine 42 and the trabecular meshwork results in a kickstand operation that tends to rotate the base portion 32 of the bypass device 30 about the attachment portion 42a of the anchor tine 42, which thus tends to press the body portion 32 securely against the inner surface of the trabecular meshwork. In this manner, the curvature of the anchoring tine as positioned against the inner surface of the trabecular meshwork acts to generate the kickstand operation that applies a holding force to aid in maintaining the implanted bypass device in place. The bypass device can be easily removed by elevating the anchor tine from the trabecular meshwork to alleviate the kickstand operation, and sliding the flexed tines backwards towards the corresponding trabecular meshwork openings. The ocular implant bypass device is therefore a minimally invasive glaucoma surgery (MIGS) device as the bypass device minimally disrupts the tissues, being implanted through a very small incision, functions without disrupting surrounding tissues, and is easy to reverse.

In exemplary embodiments, the plunger device is equipped with a holding device that holds onto the bypass device 30 by holding, gripping, or otherwise securing the bypass device at the anchor tine 42. The holding device provides more control for the surgeon in manipulating the bypass device, such as by rotation or other manipulation, in addition to the linear pushing by the plunger device, as the bypass device is advanced through the cannula for implantation. The holding device may be configured as forceps, tongs, a hook, or similar type gripping device that can hold onto the bypass device at the anchor tine. For a hook configuration, the anchor tine may be configured with a hole, ridge, slot, or similar cooperating feature that can receive the hook. When the holding device is configured as a gripping device, the device may be equipped with an automatic release mechanism, such as a spring bias, that operates to release the bypass device when the bypass device is at its final implant location.

Although the invention has been shown and described with respect to a certain embodiment or embodiments, equivalent alterations and modifications may occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein exemplary embodiment or embodiments of the invention. In addition, while a particular feature of the invention may have been described above with respect to only one or more of several embodiments, such feature may be combined with one or more other features of the other embodiments, as may be desired and advantageous for any given or particular application.

1. A bypass device for providing fluid channels through a body tissue, the bypass device comprising:
a base portion having a first end and a second end opposite from the first end;
a first flexed tine and a second flexed tine attached to the base portion and configured in a flexed position relative to the base portion, the first flexed tine being positioned closest to the first end of the base portion and the second flexed tine being positioned between the first flexed tine and the second end of the base portion;
the base portion including a first flow space present from where the first flexed tine is flexed relative to the base portion and a second flow space present from where the second flexed tine is flexed relative to the base portion, the first and second flow spaces permitting fluid flow through the bypass device; and
an anchor tine located adjacent to the second end of the base portion, and the second flexed tine is positioned between the first flexed tine and the anchor tine.

2. The bypass device of claim 1, wherein the first flexed tine and the second flexed tine each extends from the base portion at an acute angle relative to the base portion, and the anchor tine extends at an obtuse angle relative to the base portion.

3. The bypass device of claim 1, wherein each of the first flexed tine, the second flexed tine, and the anchor tine includes a respective attachment portion at which a respective tine is attached to the base portion.

4. The bypass device of claim 3, wherein each of the first flexed tine, the second flexed tine, and the anchor tine includes a respective curved portion that extends from the respective attachment portion, and a respective body that extends from the respective curved portion.

5. The bypass device of claim 4, wherein the curved portion of the first flexed tine and the curved portion of the second flexed tine each extends from the base portion at an acute angle relative to the base portion, and the curved portion of the anchor tine extends at an obtuse angle relative to the base portion.

6. The bypass device of claim 5, wherein the obtuse angle of the curved portion of the anchor tine acts to generate a holding force to aid in maintaining the bypass device in place after implantation.

7. The bypass device of claim 5, wherein the obtuse angle of the curved portion of the anchor tine is from 150° to 170°.

8. The bypass device of claim 1, wherein the flexed position of each of the first flexed tine and the second flexed tine creates a flat spring with an associated spring force.

9. The bypass device of claim 1, wherein each of the first flexed tine, the second flexed tine, and the anchor tine includes a respective arrow end.

10. The bypass device of claim 1, wherein the base portion has a curvature that approximates a curvature of iridocorneal angle structures.

11. A method of performing a minimally invasive glaucoma surgery (MIGS) for treating glaucoma comprising the steps of:
providing a bypass device for bypassing a drainage system of an eye including a Schlemm's canal, trabecular meshwork, and collector channels, the bypass device comprising:
a base portion having a first end and a second end opposite from the first end;
a first flexed tine and a second flexed tine attached to the base portion configured in a flexed position relative to the base portion, the first flexed tine being positioned closest to the first end of the base portion and the second flexed tine being positioned between the first flexed tine and the second end of the base portion;
the base portion including a first flow space present from where the first flexed tine is flexed relative to the base portion and a second flow space present from where the second flexed tine is flexed relative to the base portion, the first and second flow spaces permitting fluid flow through the bypass device; and
an anchor tine located adjacent to the second end of the base portion, and the second flexed tine is positioned between the first flexed tine and the anchor tine;
providing an introducer comprising a cannula and an advancing plunger that is threaded into the cannula;
locating the bypass device within the cannula at an initial retracted position with the advancing plunger being positioned in the cannula rearward relative to the bypass device;
forming a guide hole to access the Schlemm's canal at the trabecular meshwork of the eye;
inserting the cannula with the bypass device and advancing plunger through the guide hole and positioning the cannula adjacent to the trabecular meshwork;
operating the introducer to operate the advancing plunger to advance the bypass device through the cannula from the initial retracted position to an intermediate position to expose the first and second flexed tines from the cannula;
puncturing the trabecular meshwork with ends of the first and second flexed tines and forcing the flexed tines through the trabecular meshwork;
operating the introducer to operate the advancing plunger to further advance the bypass device through the cannula from the intermediate position to an extended position;
wherein as the bypass device advances from the initial retracted position to the extended position, the flexed tines tear an elongated flow hole through trabecular meshwork, such that when the bypass device reaches the extended position the elongated flow hole is aligned with the flow spaces of the base portion of the bypass device, and aqueous humor is permitted to flow through the flow spaces of the bypass device, and then through the aligned elongated flow hole through the trabecular meshwork and into the Schlemm's canal so that the aqueous humor can drain; and
retrieving the cannula with the advancing plunger while leaving the bypass device in place, wherein when the bypass device has reached the extended position and the cannula is removed, the anchor tine is positioned against an inner surface of the trabecular meshwork, and a curvature of the anchoring tine as positioned against the inner surface of the trabecular meshwork acts to generate a holding force to aid in maintaining the bypass device in place.

12. The MIGS method of claim 11, wherein the bypass device is configured according to any of claims 2-10.

13. The MIGS method of claim 11, wherein the introducer further includes a first slider and a second slider, the method further comprising operating the first slider to operate the advancing plunger to advance the bypass device from the initial retracted position to the intermediate position and operating the second slider to operate the advancing plunger advance the bypass device from the intermediate position to the extended position.

14. The MIGS method of claim 11, wherein the flexed position of each of the first flexed tine and the second flexed tine creates a flat spring with an associated spring force, and puncturing the trabecular meshwork comprises applying pressure with the ends of the first and second flexed tines against the trabecular meshwork to compress the first and second flexed tines against the spring force, such that the spring force aids the to push the ends of the first and second flexed tines through the trabecular meshwork.

15. The MIGS method of claim 11, wherein when the bypass device is in the extended position, the anchor tine has a curvature that extends along an inner surface of the trabecular meshwork in opposition to the curvature of the inner surface, such that a force interaction between the anchor tine and the inner surface of the trabecular meshwork tends to rotate the base portion of the bypass device to press the base portion against the inner surface of the trabecular meshwork to aid in maintaining the bypass device in place.

16. The MIGS method of claim 11, wherein the introducer further includes a curved stent attached to a forward end of the cannula, and the bypass device passes through the curved stent when the introducer is operated.

17. The MIGS method of any of claim 11, further comprising performing a visualization technique to locate clogged portions of the drainage system, wherein the bypass device is aligned relative to the trabecular meshwork such that the first and second flexed tines are positioned at or adjacent to respective clogged portions of the trabecular meshwork as determined by the visualization technique.

18. The MIGS method of claim 11, further comprising injecting a viscoelastic substance through the cannula and into the Schlemm's canal to expand the Schlemm's canal to aid positioning the bypass device.

19. The MIGS method of claim 11, further comprising introducing one or more substances into the Schlemm's canal through the cannula, wherein the one or more substances include one or more of a glaucoma medication, an anti-inflammatory agent, and an antibiotic releasing pellet.

20. The MIGS method of claim 11, wherein the advancing plunger includes a holding device, the MIGS method further comprising holding the bypass device at the anchor tine with the holding device while advancing the bypass device with the advancing plunger.
